(19) United States
(12) Patent Application Publication
(10) Pub. No.: US 2013/0231625 A1
(43) Pub. Date: Sep. 5, 2013

(54) ADJUSTABLE PANT-LIKE DISPOSABLE UNDERGARMENT WITH FULLY SEVERED FRONT PANEL

(71) Applicants: Kimberly L. Ellefson, Oshkosh, WI (US); Kimberly M. Downs, Oshkosh, WI (US); Suzanne M. Schmoker, Oshkosh, WI (US)

(72) Inventors: Kimberly L. Ellefson, Oshkosh, WI (US); Kimberly M. Downs, Oshkosh, WI (US); Suzanne M. Schmoker, Oshkosh, WI (US)

(21) Appl. No.: 13/412,482

(22) Filed: Mar. 5, 2012

(51) Int. Cl. A61F 13/505

(52) U.S. Cl. 604/385.11

(57) ABSTRACT

A pre-fastened adjustable pant-like disposable absorbent undergarment including a fully severed front panel is disclosed. The front panel includes a first side section, a second side section, and a middle section, wherein the front body panel is completely severed between the first side section and the middle section, and wherein the front body panel is completely severed between the second side section and the middle section. In addition, a pair of attachment members are secured to the side sections and releasably attached to the middle section. The pre-fastened adjustable pant-like disposable absorbent undergarment also includes a back body panel, and an absorbent assembly secured to said front and back body panels, and said front and back body panels being joined together by a pair of side seams to form a waist opening and a pair of leg openings.

### BACKGROUND

Pant-like disposable undergarments for absorbing human discharges can appear similar in size and shape to regular cloth underwear which is designed to be laundered and reused two or more times. A disposable absorbent undergarment is intended to be worn by persons, including infants, toddlers, or adults, and is designed for a single or temporary use and is meant to be disposed of after being used once instead of being laundered or dry cleaned for re-use. Some examples of disposable undergarments include infant diapers, training pants, adult incontinence garments, feminine pants, etc.

Some pant-like disposable absorbent undergarments manufactured today resemble regular cloth underwear in that they have a waist opening and a pair of leg openings. Such pant-like disposable absorbent undergarments can be pulled up around the torso of a wearer in a similar fashion as regular cloth underwear. Still other pant-like disposable absorbent undergarments have an open or flat configuration and are designed to be placed adjacent to a wearer's torso and then rely upon one or more attachment tabs or fasteners to secure the undergarment around the wearer's torso. This design is beneficial for bed bound users who may be immobile or for children who need assistance in securing the undergarment in place. Still other adjustable, pant-like absorbent undergarments contain attachment means for opening and closing the waist opening after the undergarment has been positioned around the wearer's torso. The adjustable undergarment has an advantage in that the wearer does not have to remove outer clothing in order to check the status of the undergarment or to remove the undergarment from their body.

One example of such an adjustable, pant-like disposable absorbent undergarment includes a pair of lines of weakness that a user must break to enable adjusting the fit of the undergarment. The lines of weakness usually extend from the waist opening to one of the leg openings and are designed to be broken either prior to positioning the undergarment around the user's torso or while the undergarment is already positioned about the wearer's torso. A pair of attachment members is then utilized to refasten the undergarment so that it is snug about the wearer's torso.

It has been found that a major portion of each of the lines of weakness is visually hidden and some users cannot see them and thereby do not know that they are present. In addition, each line of weakness may be ergonomically hard to tear open by older adults, some of who may be suffering from arthritis. In addition, options that require tearing often indicate to users that the product is damaged or of poor quality when torn.

In addition, with certain refastenable garments, such as adult incontinence underwear and enuresis pants, it is important that the garments look and feel as much as possible like “regular” underwear to promote an improved sense of normalcy to the wearer who suffers from incontinence or enuresis. Accordingly, there is a desire in certain cases to incorporate fastening systems in garments in such a way that the fastening system is relatively discreet in order to make the garment more like “regular” cloth underwear. One possible approach is to provide at least one garment-like, refastenable seam, provided by one or more strips of mechanical fastening material, such as a hook-and-loop style fastener. However, one potential drawback to such designs is that a garment-like refastenable seam may be difficult to detect and difficult to manipulate by the user of the product. In particular embodiments, when a fastener which lies relatively close to an edge of the base substrate to which it is applied, it can in some cases be difficult to locate the edge of the refastenable seam, and, once located, it can in some cases be difficult to grasp the edge of the refastenable seam so as to disengage the seam to allow the product to be opened and inspected or removed.

As a result, an adjustable, pant-like disposable absorbent undergarment that includes a fully severed front panel and more readily apparent and more easily accessible fastener mechanisms is needed.

### SUMMARY

Generally, a pre-fastened adjustable pant-like disposable absorbent undergarment including a fully severed front panel is disclosed. The front panel includes a first side section, a second side section, and a middle section, wherein the front body panel is completely severed between the first side section and the middle section, and wherein the front body panel is completely severed between the second side section and the middle section. In addition, a first attachment member is secured to the first side section and attached to the middle section. The first attachment member is releasably attached to said first side section or middle section. A second attachment member is secured to the first side section and attached to the middle section, the second attachment member is releasably attached to said second side section or middle section. The pre-fastened adjustable pant-like disposable absorbent article also includes a back body panel, and an absorbent assembly secured to said front and back body panels, and said front and back body panels being joined together by a pair of side seams to form a waist opening and a pair of leg openings.

Each of the attachment members extends almost the entire length of the fully severed portions. By extending substantially the entire length of and bridging the fully severed portions, the attachment members cover the fully severed portions of the undergarment and provide a more underwear like appearance. Desirably, the first attachment member and second attachment member extend at least 75% of the distance between the waist end and the leg opening. More desirably, the first attachment member and second attachment member completely extend at least 95% of the distance between the waist end and the leg opening.

In exemplary embodiments, the front body panel contains a pair of attachment members which facilitate inspection and possible removal of the undergarment from a wearer's body without requiring the wearer to remove any outer clothing. Desirably, in some embodiments, the first attachment member and the second attachment member are located near the midpoint between the side seam and the cross-sectional midpoint of the front panel. Desirably, the first attachment member and second attachment member is located between 30% and 70% of the distance between the side seam and the cross-sectional midpoint of the front panel. More desirably, the first attachment member and second attachment member are located between 35% and 45% of the distance between the side seam and the cross sectional midpoint of the front panel.

### BRIEF DESCRIPTION

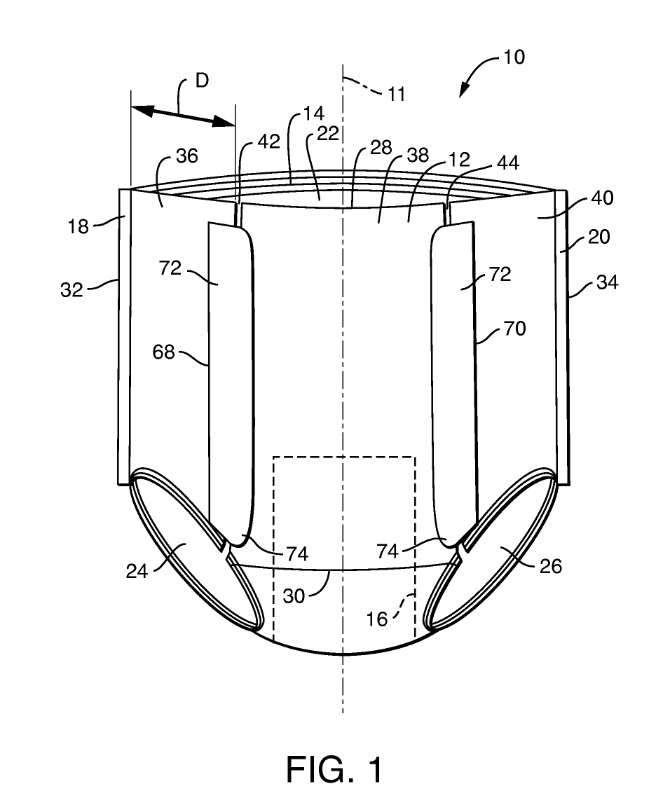

FIG. 1 depicts a front perspective view of one example of a garment, the garment shown in a pre-fastened, pant-like configuration.

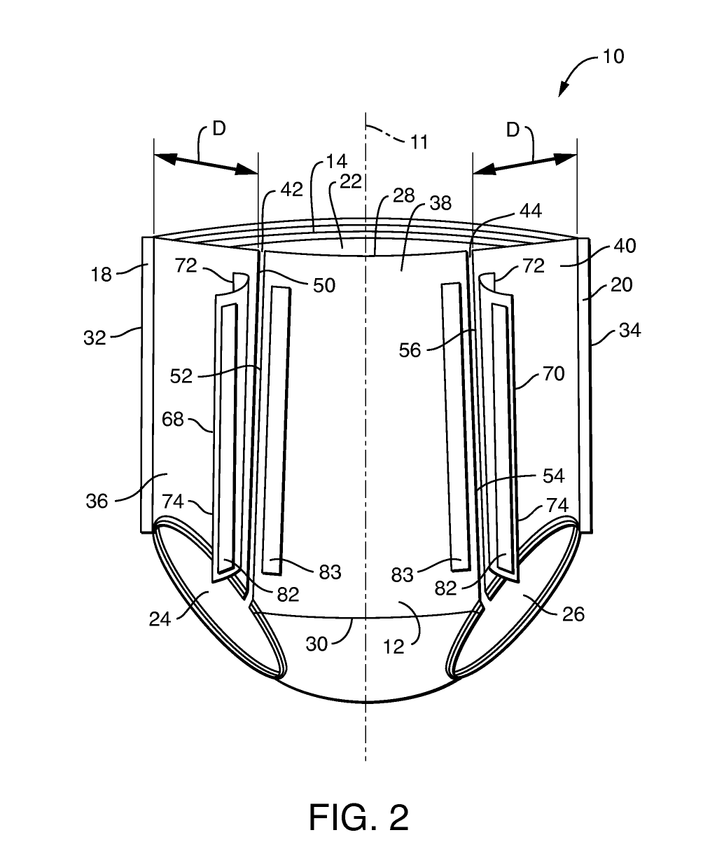

FIG. 2 depicts a front perspective view of one example of a garment, the garment shown in a shown in an unfastened, pant-like configuration.

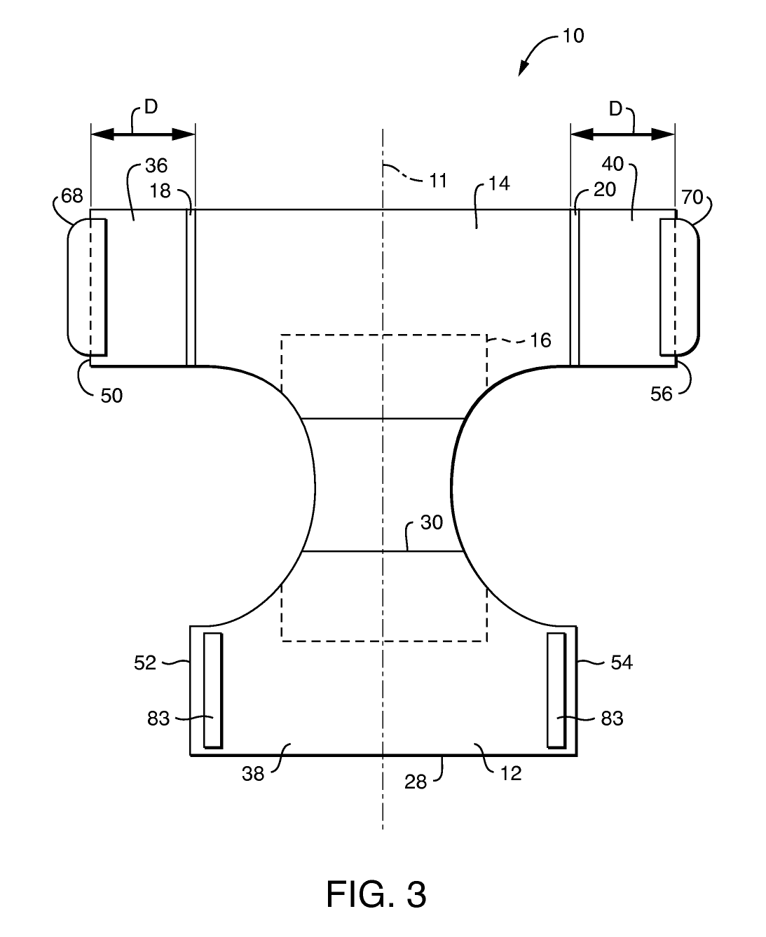

FIG. 3 depicts a plan view of the garment of FIG. 1, the garment shown in an unfastened, laid-open, relaxed configuration.

### DETAILED DESCRIPTION

Generally, a pre-fastened adjustable pant-like disposable absorbent undergarment including a fully severed front panel is disclosed. Reference to the Figures shall be made in describing various embodiments. It should be noted that the embodiments depicted in the Figures and described herein are merely representative examples. The various embodiments are suitable for use in conjunction with disposable absorbent undergarments such as refastenable adult incontinence underwear, pre-fastened disposable diapers, refastenable disposable training pants or swim pants, refastenable disposable enuresis garments, and the like. For illustration purposes, various embodiments shall be described in conjunction with refastenable incontinence or enuresis underwear.

Within the context of this specification, each term or phrase below will include the following meaning or meanings.

“Attached” refers to the joining, adhering, bonding, connecting, or the like, of two elements. Two elements will be considered to be attached together when they are attached directly to one another or indirectly to one another, such as when each is directly attached to intermediate elements.

“Disposable” refers to articles which are designed to be discarded after a limited use rather than being laundered or otherwise restored for reuse.

“Elastomeric” refers to a material or composite which can be elongated by at least 50% of its relaxed length and which will recover, upon release of the applied force, at least 20% of its elongation. It is generally preferred that the elastomeric material or composite be capable of being elongated by at least 100%, more preferably by at least 200%, of its relaxed length and recover, upon release of an applied force, at least 50% of its elongation.

“Longitudinal” and “transverse” have their customary meaning, as indicated by the longitudinal and transverse axes depicted in the Figures. The longitudinal axis lies in the plane of the article and is generally parallel to a vertical plane that bisects a standing wearer into left and right body halves when the article is worn. The transverse axis lies in the plane of the article generally perpendicular to the longitudinal axis.

These terms may be defined with additional language in the remaining portions of the specification.

Referring to FIGS. 1-3, an adjustable pant-like disposable absorbent undergarment 10 having a longitudinal axis 11 is shown. The adjustable pant-like disposable absorbent undergarment 10 is designed to absorb liquid, semi-solid and/or solid waste discharged from a human being. A “disposable absorbent undergarment” as used herein is an article that is intended to be worn by persons, including infants, toddlers or adults, which is designed for a single or temporary use and is meant to be disposed of after being used once instead of being laundered or dry cleaned for re-use. The adjustable pant-like disposable absorbent undergarment 10 is designed to absorb and/or retain one or more bodily discharges of waste material such as urine, perspiration, excrement, feces, menses, menstrual fluid, as well as other liquid and/or solid waste.

The adjustable pant-like disposable absorbent undergarment 10 includes a front body panel 12, a back body panel 14 and an absorbent assembly 16 secured to the front and back body panels, 12 and 14 respectively. The front and back body panels, 12 and 14 respectively, are joined together by a pair of seams 18 and 20 to form a waist opening 22 and a pair of leg openings 24 and 26.

The front body panel 12 and/or the back body panel 14 can be formed from a single piece of material or they can be formed as a laminate consisting of two or more layers. The layers of the laminate can be of the same material or different material. In one embodiment, a laminate is formed from a first layer and a second layer. Sandwiched between the first and second layers are two or more elastic strands. Desirably, from two to about a hundred elastic strands can be utilized in either the front or back body panels, 12 and 14, depending upon the overall size of each panel. The elastic strands 56 can be formed from LYCRA, or a similar material. The diameter and/or cross-sectional configuration of the elastic strands 56, the decitex (weight in grams per 10,000 meters) of the elastic strands, and the tension imparted into the elastic strands 56 can all be varied to suit one's particular product needs. The number of elastic strands present in the front body panel 12 can be less than, equal to or greater than the number of elastic strands present in the back body panel 14. The exact number of elastic strands that are utilized should be sufficient to ensure that the disposable absorbent undergarment 10 snuggly conforms to the wearer's torso.

The elastic strands can be coated with an adhesive. By adhesively coating each of the elastic strands, instead of slot coating a major portion of the inner surface of at least one of the first and second layers, softer front and back body panels, 12 and 14 respectively, can be obtained. Wearers of disposable absorbent undergarments prefer a product that has a softer feel since it is more underwear like.

In other embodiments, the elastomeric nonwoven material comprises an elastomeric film sandwiched between two nonwoven facing layers. U.S. Pat. No. 7,803,244 to Siqueira et al., hereby incorporated by reference, discloses particular examples of elastomeric nonwoven composites suitable for use in the adjustable undergarment described herein.

It should be noted that the front and/or back body panels, 12 and/or 14 respectively, can be formed from a breathable or a non-breathable material. Desirably, the front and back body panels, 12 and 14 respectively, are formed from a breathable material or a material that is treated or processed to be breathable. Spunbond and bonded carded webs are two breathable materials that work well as front and back body panels, 12 and 14 respectively, in disposable absorbent undergarments. Bonded carded webs are produced and commercially sold by a variety of vendors. Other materials that can be used to form the front and back body panels, 12 and 14 respectively, include woven and non-woven materials formed from natural or synthetic fibers; polyolefins, such as polypropylene or polyethylene; thermoplastic films; as well as other materials known to those skilled in the art. A metallocene polypropylene works very well since it has a soft feel and can be easily ultrasonically bonded to itself.

The front body panel 12 includes a waist edge 28, a crotch edge 30 and a pair of side edges 32 and 34. In a three piece construction wherein an absorbent assembly 16 is secured between the front body panel 12 and the back body panel 14, the crotch edge 30 is well defined. In absorbent undergarments of a different construction, the crotch edge 30 can be an imaginary line transversely drawn between the pair of leg openings 24 and 26 at a location where one considers the front body panel 12 to end. The exact size and configuration of the front body panel 12 can vary to suit one's particular needs. The front body panel 12 has a first side section 36, a middle region 38 and a second side section 40. The front body panel 12 is completely severed between both the first side section 36 and the middle section 38 and the second side section 40 and the middle section 38 creating a first fully severed region, or gap, 42 and a second fully severed region 44. The first side section 36 is aligned adjacent to the side edge 32 at its leading edge forming the seam 20 and the second side section 40 is aligned adjacent to the side edge 34 at its leading edge forming the seam 18. The middle section 38 has a first edge and a second edge and is located between the first and second sections, 36 and 40 respectively. The terminal edge 50 of the first side section is aligned adjacent to the first edge 52 of the middle section 38 and the terminal edge 56 of the second side section 40 is aligned adjacent to the second edge 54 of the middle section 38. As depicted, the middle section 38 is centrally located and is bifurcated by the longitudinal axis 11 at a midpoint of the front body panel 12.

The first and second fully severed regions, 42 and 44, can be linear or non-linear in configuration. In FIGS. 1-3, the first and second fully severed region, 42 and 44, are shown having a linear or straight configuration. The first and second fully severed regions, 42 and 44, extend longitudinally from approximately the waist edge 28 down to approximately one of the leg openings 24 or 26. The first and second fully severed regions, 42 and 44, can be aligned parallel to the longitudinal axis 11 or be angled thereto. For example, the first and second fully severed cuts, 42 and 44, can be tapered relative to the longitudinal axis 11, if desired. In addition, the fully severed cuts, 42 and 44, can also be curved.

Still referring to FIGS. 1-3, each of the first and second fully severed regions, 42 and 44, extend from approximately the waist edge 28 of the front body panel 12 to one of the pair of leg openings 24 and 26. Another way of describing this is to say that the first and second fully severed regions, 42 and 44, extend from approximately the waist edge 28 of the front body panel 12 to approximately the crotch edge 30 of the front body panel 12.

Many adjustable, pant-like disposable absorbent undergarments include a pair of lines of weakness to break to allow for the individual to adjust the size of the pants. This type of adjustable disposable undergarment including the lines of weakness requires an amount of force needed to break the lines of weakness. The pre-fastened adjustable, pant-like disposable absorbent undergarments described herein have the first and second fully severed regions, 42 and 44, that require no force to break allowing for easier use by an elderly individual, caretaker or parent caring for a child.

Still referring to FIGS. 1-3, each of the first and second fully severed regions, 42 and 44, is shown being aligned parallel to one of the pair of seams 18 and 20. Such an arrangement provides for an aesthetically pleasing appearance to the front body panel 12.

Desirably, in the pre-fastened condition, the first side sections 36 and the middle section 38 of front body panel 12 do not overlap, and the second side section 40 and the middle section 38 of the front body panel 12 do not overlap in the severed regions 42, 44. In one exemplary embodiment, a first gap may be formed in the first severed region 42 between the first side section 36 and the middle section 38 of front body panel 12 in the pre-fastened condition. In this embodiment, a second gap may be formed in the first severed region 44 between the second side section 40 and the middle section 38 of the front body panel 12 in the pre-fastened condition. In another exemplary embodiment, a terminal or outer edge 50 of the first side section 36 abuts against the first edge 52 of the middle section 38 of front body panel 12 in the pre-fastened condition. In this embodiment, a terminal or outer edge 56 of the second side section 40 abuts against the second edge 54 of the middle section 38 of the front body panel 12 in the pre-fastened condition. In another embodiment, a gap may be formed between one side section and the middle section and the other side section and middle section abut against each other. If a gap is formed between the side sections 36 and 38, and the middle section 38, the gap will desirably be less than 10 mm.

Referring again to FIGS. 1-3, the front body panel 12 further includes a pair of attachment members 68 and 70. Each attachment member 68 and 70 includes a first portion 72 and a second portion 74. The first portion 72 of the attachment member 68 can be permanently secured to the first region 36 of the front body panel 12 and the first portion 72 of the other attachment member 70 can be permanently secured to the third region 40 of the front body panel 12.

In the embodiment shown in FIGS. 1-3, the first portions 72 can be securely attached using an adhesive, heat, pressure, a combination of heat and pressure, an ultrasonic bond, a chemical bond or by other means known to those skilled in the art.

Each of the second portions 74 of the attachment members 68 and 70 are depicted as extending almost the entire length of the fully severed portions 42 and 44. This means at least 50% of the distance between the waist end and the leg opening are covered by the attachment member. Desirably, the first attachment member and second attachment member extend at least 75% of the distance between the waist end and the leg opening. More desirably, the first attachment member and second attachment member completely extend at least 95% of the distance between the waist end and the leg opening. The second portions 74 of the attachment member 68 can bridge across the first fully severed region 42 and the second portion 74 of the other attachment member 70 can bridge across the second fully severed region 44. By extending substantially the entire length of and bridging the fully severed portions, 42 and 44, the fasteners cover the fully severed portions and provide a more underwear like appearance. The second portions 74 of the attachment members 68 and 70 can be releasably attached to the middle section 38 of the front body panel 12.

Alternatively, it should be evident to those skilled in the art that the first portion 72 of each of the attachment members 68 and 70 could be permanently attached to the middle section 38. In this embodiment, the second portions 74 of the attachment members 68 and 70 can be releasably attached to the side sections of the front body panel 12. In an alternative embodiment, both the first portion 72 and the second portion 74 may be releasably attached. The fastener may be constructed of a non-extensible or an elastomeric material.

Referring now to FIGS. 2 and 3, each of the second portions 74 of the attachment members 68 and 70 has an inner surface 80 that contains a fastener 82. The fastener 82 can be a mechanical fastener. In FIGS. 2 and 3, the mechanical fastener 82 is shown as a plurality of fine hooks, such as VELCRO hooks. VELCRO is a trademark of Velcro USA, Inc. (Manchester, N.H.). The hooks are designed to easily engage and be removed from a material wherein the material has a loose weave pattern or the fibers forming the material will allow the hooks to be attached to them. The mating material is commonly referred to as the loop member of a hook and loop fastener. The middle section 38 of the front body panel 12 may be formed of such a material. When the hooks engage into the middle section 38, a secure but releasable fastener is formed. The hooks can be easily removed from the loop material by pulling the edge of the fastener outward away from the middle section 38. Alternatively, as illustrated in FIGS. 2 and 3, a fastening component 83 such as a loop material or loose fibers that may be placed anywhere onto the middle section 38 to facilitate the fastening component. Therefore, hook and loop fasteners are referred to as being releasable and can be fastened and released several times. In alternative embodiments, the mechanical fastener 82 constructed of hooks may be placed on to the middle section 38 of the front body panel 12. In this embodiment, the fastening component 83 constructed of a loop material or loose fibers would be placed on the attachment member 68, 70. Both the mechanical fastener 82 and the fastening component 83 may be integral or separately attached.

Referring again to FIGS. 1-3, one can see that FIG. 1 depicts the pair of attachment members 68 and 70 being securely fastened to the middle section 38 of the front body panel 12. If the wearer of the disposable absorbent undergarment 10 or a caregiver wishes to inspect the undergarment 10, he or she would open the pair of attachment members 68 and 70 to the position shown in FIG. 2. Since the first and fully severed portions, 42 and 44 are already broken, the middle section 38 of the disposable absorbent undergarment 10 can be easily moved outward away from the wearer's torso. The wearer can then inspect the absorbent assembly 16 to see if it needs to be changed. If so, the disposable absorbent undergarment 10 can be removed from about the wearer's torso and be replaced by another undergarment. If the absorbent assembly 16 is still capable of accepting additional body fluid, the middle section 38 is moved back against the wearer's torso and the pair of attachment members 68 and 70 is refastened to the middle section 38.

The horizontal distance to each of the first and second fully severed regions, 42 and 44, and corresponding first and second attachment members, 68 and 70, as located relative to the pair of side seams 18 and 20 can also vary. A distance “D” is depicted in FIGS. 1-3 which represents the distance between each of the pair of seams 18 and 20 and the corresponding first and second fully severed regions, 42 and 44 respectively. One can increase the distance “D” by moving the first and second fully severed regions, 42 and 44 respectively, away from the pair of seams 18 and 20. When one increases the distance “D”, one may find that it is easier for the wearer of the disposable absorbent undergarment 10 to visually see and identify the first and second fully severed regions, 42 and 44, when user looks down at the front body panel 12. In some embodiments, the first attachment member 68 and the second attachment member 70 are located near the midpoint between the side seam, 18 and 20, and the cross-sectional midpoint 11 of the front panel 12. Desirably, the first attachment member 68 and second attachment member 70 are located between 30% and 70% of the distance between the side seam, 18 and 20, and the cross-sectional midpoint 11 of the front panel 12. More desirably, the first attachment member 68 and second attachment member 70 are located between 35% and 45% of the distance between the side seam, 18 and 20, and the cross-sectional midpoint 11 of the front panel 12. A manufacturer is free to vary the distance “D” to best suit the size and shape of a particular disposable absorbent undergarment 10 to make sure that the fastener is on the front of the product rather than the side of the product. If the fully severed regions are not parallel to the side seam, the midpoint of the fully severed regions in the longitudinal direction may be used to measure “D”.

Placement near the midpoint between the side seam and the cross-sectional midpoint of the front body panel facilitates enhanced donning and removal when experiencing a reduced range of motion as well as locating the fastener in a location which is easier for the consumer to see.

The placement of the attachment members 68 and 70 in the front of the product optimizes the ability of the user to fasten the product, while minimizing potential pop-opens during use due to movement of the legs of the user. The placement of the fasteners can be measured in both an unstretched (out of the bag) state or stretched (as in use).

It should be understood that various other embodiments, modifications, and equivalents to the embodiments of the absorbent article described herein which, after reading the description herein, may suggest themselves to those skilled in the art without departing from the scope and spirit of the present claims.

What is claimed is:

1. A body panel for a pre-fastened adjustable pant-like disposable absorbent undergarment comprising:
a. a first side section, a second side section, and a middle section, wherein the front body panel is completely severed between the first side section and the middle section, and wherein the front body panel is completely severed between the second side section and the middle section;
b. a first attachment member being attached to the first side section and attached to the middle section, the first attachment member being releasably attached to either said first side section or middle section; and
c. a second attachment member being attached to the second side section and attached to the middle section, the second attachment member being releasably attached to either said second side section or middle section.

2. The body panel of claim 1 wherein the first side section and the middle section of the front body panel do not overlap, and the second side section and the middle section of the front body panel do not overlap.

3. The body panel of claim 1 wherein a first gap exists between the first side section and the middle section of the front body panel in the pre-fastened condition, and wherein a second gap exists between the second side section and the middle section of the front body panel in the pre-fastened condition.

4. The body panel of claim 1 wherein a terminal edge of the first side section abuts against the first edge of the middle section of the front body panel in the pre-fastened condition, and wherein a terminal edge of the second side section abuts against the second edge of the middle section of the front body panel in the pre-fastened condition.

5. The body panel of claim 1 wherein the first attachment member and the second attachment member extend at least 75% of the distance between the waist end and the leg opening.

6. The body panel of claim 1 wherein the first attachment member and the second attachment member completely extend at least 95% of the distance between the waist end and the leg opening.

7. The body panel of claim 1 wherein the first attachment member and the second attachment member are located near the midpoint between the side seam and the cross-sectional midpoint of the front panel.

8. The body panel of claim 7 wherein the first attachment member and the second attachment member are located between 30% and 70% of the distance between the side seam and the cross-sectional midpoint of the front panel.

9. The body panel of claim 8 wherein the first attachment member and the second attachment member are located between 35% and 45% of the distance between the side seam and the cross-sectional midpoint of the front panel.

10. A pre-fastened adjustable pant-like disposable absorbent undergarment comprising:
a. a front body panel having a first side section, a second side section, and a middle section, wherein the front body panel is completely severed between the first side section and the middle section, and wherein the front body panel is completely severed between the second side section and the middle section;
b. a first attachment member being attached to the first side section and attached to the middle section, the first attachment member being releasably attached to either said first side section or middle section;
c. a second attachment member being attached to the first side section and attached to the middle section, the second attachment member being releasably attached to either said second side section or middle section;
d. a back body panel; and
e. an absorbent assembly secured to said front and back body panels, and said front and back body panels being joined together by a pair of side seams to form a waist opening and a pair of leg openings.

11. The pre-fastened adjustable pant-like disposable undergarment of claim 10 wherein the first side section and the middle section of the front body panel do not overlap, and the second side section and the middle section of the front body panel do not overlap.

12. The pre-fastened adjustable pant-like disposable undergarment of claim 10 wherein a first gap exists between the first side section and the middle section of the front body panel, and wherein a second gap exists between the second side section and the middle section of the front body panel.

13. The pre-fastened adjustable pant-like disposable undergarment of claim 10 wherein a terminal edge of the first side section abuts against the first edge of the middle section of the front body panel, and wherein a terminal edge of the second side section abuts against the second edge of the middle section of the front body.

14. The pre-fastened adjustable pant-like disposable undergarment of claim 10 wherein a terminal edge of one of the first or second side sections abuts against the edge of the middle section of the front body panel and the first gap exists between one of the first or second side sections and the middle section of the front body panel.

15. The pre-fastened adjustable pant-like disposable undergarment of claim 10 wherein the first attachment member and the second attachment member extend at least 75% of the distance between the waist end and the leg opening.

16. The pre-fastened adjustable pant-like disposable undergarment of claim 15 wherein the first attachment member and the second attachment member completely extend at least 95% of the distance between the waist end and the leg opening.

17. The pre-fastened adjustable pant-like disposable undergarment of claim 10 wherein the first attachment member and the second attachment member are located near the midpoint between the side seam and the cross sectional midpoint of the front panel.

18. The pre-fastened adjustable pant-like disposable undergarment of claim 16 wherein the first attachment member and the second attachment member are located between 30% and 70% of the distance between the side seam and the cross sectional midpoint of the front panel.

19. The pre-fastened adjustable pant-like disposable undergarment of claim 17 wherein the first attachment member and the second attachment member are located between 35% and 45% of the distance between the side seam and the cross sectional midpoint of the front panel.
